(19) United States
(12) Patent Application Publication
(10) Pub. No.: US 2026/0144582 A1
(43) Pub. Date: May 28, 2026

(54) MULTI-LUMEN CANNULA AND METHODS OF USING OF SAME

(71) Applicant: ARTHREX, INC., Naples, FL (US)

(72) Inventors: John David Adams, JR., Greenville, SC (US); Alexander DelMonaco, Chelmsford, MA (US); Manish Thapa, North Grafton, MA (US); Brittany Foslien, Naples, FL (US); Victoria Triaga, Naples, FL (US)

(21) Appl. No.: 19/401,884

(22) Filed: Nov. 26, 2025

Related U.S. Application Data
(60) Provisional application No. 63/725,935, filed on Nov. 27, 2024.

(51) Int. Cl. A61B 17/88; A61B 17/56; A61B 17/72; A61B 17/90

(52) U.S. Cl. CPC A61B 17/8872; A61B 17/7291; A61B 17/90; A61B 2017/564; A61B 17/7266

(57) ABSTRACT

Disclosed herein are components, systems, and methods to guide and support temporary bone fixators on either side of an implant. The temporary bone fixators are securable within a fragment of a fractured bone enabling a force to be applied to the fragment, via the temporary bone fixators, to move the fragment relative to another fragment of the fractured bone. A multi-lumen cannula includes a lumen for one or more of the temporary bone fixators and a lumen for one or more permanent bone fixators that secure the implant relative to the fractured bone. The multi-lumen cannula may have a body with equal portions divided by a plane of symmetry, enabling the multi-lumen cannula to be reversibly inserted into a recess of a targeting guide that aligns the lumens of the multi-lumen cannula relative to the implant.

### CROSS-REFERENCE TO RELATED APPLICATION

This application claims the benefit of Provisional Application No. 63/725,935, filed Nov. 27, 2024, which is hereby incorporated in its entirety herein.

### BACKGROUND

Bone fractures are a common medical condition both in the young and old segments of the population. One current treatment of bone fractures includes surgically resetting the fractured bone. After the surgical procedure, the fractured area of the body (e.g., a limb in which the fractured bone is located) is often placed in an external cast for an extended period of time to ensure that the fractured bone heals properly. This can take several months for the bone to heal and for the patient to remove the cast before resuming normal activities.

In some instances, an intramedullary (IM) nail is used to align and stabilize the fracture. In that instance, a metal rod is placed inside a canal of a bone and fixed in place, typically at both ends. Placement of conventional IM nails typically require access collinear with a center line of the IM canal. For example, a fibula fracture may be treated by insertion of an IM nail. During insertion the IM nail enters a distal fragment of the fibula, is advanced across a fracture site, and enters a proximal fragment of the fibula.

Movement of the IM nail across the fracture site may result in movement of the distal fragment toward the proximal fragment, thereby shortening the fibula compared to its pre-fracture length. Maintaining the pre-fracture length of the fibula after the IM nail is inserted and prior to insertion of bone fixators to secure the IM nail to the distal and proximal fragments of the fibula may improve outcomes for treatment of fibular fractures.

### BRIEF SUMMARY

Some embodiments described herein provide a multi-lumen cannula that establishes respective pathways for each of a pair of temporary bone fixators (e.g., K-wires, guide wires, pins, etc.) into a fragment of a fractured bone. The multi-lumen cannula may be receivable in a targeting guide secured to an implant such that the pair of temporary bone fixators bracket (e.g., pass on either side of) the implant. The pair of temporary bone fixators may be inserted into the fragment to secure the targeting guide and the implant relative to the fragment. A force may then be applied to the targeting guide to move the fragment (e.g., relative to another fragment of the fractured bone) so as to achieve a desired length and/or desired orientation. The pair of temporary bone fixators may then be further advanced (e.g., into a second bone) to secure the fragment. The implant may then be secured to both fragments of the fractured bone via one or more permanent bone fixators (e.g., via bone screws, deployable talons, sutures, flexible wires, etc.).

The multi-lumen cannula may further establish respective pathways for one or more of the permanent bone fixators through a fixation hole of the implant and into the fragment of the fractured bone. When the implant is secured to both fragments of the fractured bone, the pair of temporary bone fixators may be removed from the fragment of the fractured bone. The multi-lumen cannula may be removed from the targeting guide, and the targeting guide may be decoupled from the implant. Upon completion of such a procedure using the multi-lumen cannula, only the implant and the permanent bone fixators remain secured to the fractured bone.

Insertion of an IM nail across a fracture site and into the two fragments of a fractured bone may push the two fragments together. This relative movement of the fragments may result in the fractured bone having a reduced length compared to its pre-fracture condition. Securing the fragments (e.g., via an IM nail) with the fragments pushed together may result in complications or undesired outcomes. Thus, embodiments of the multi-lumen cannula described herein may be used as a fracture reduction sleeve to provisionally secure the IM nail relative to one of the fragments of the fractured bone. A force may then be applied to the secured fragment (e.g., via a targeting guide with a recess through which the multi-lumen cannula is inserted) to move the secured fragment relative to an unsecured fragment of the fractured bone.

The multi-lumen cannula may include a first lumen, a second lumen, and a third lumen that each extend through a body of the multi-lumen cannula along respective axes. The first axis of the first lumen may be spaced apart from the second axis of the second lumen by a width sufficient to accommodate a width of the IM nail therebetween. The third axis of the third lumen may be aligned with a bone fixation hole of the IM nail. The first and second lumen being spaced apart so as to bracket the IM nail advantageously secure the multi-lumen cannula relative to the fragment of the fractured bone such that application of the force results in little to no rotational movement (e.g., movement other than axial movement parallel to the IM canal) of the of the fragment.

Some IM nails may be secured to a fragment (e.g., a distal fragment) of the fractured bone (e.g., a fibula) via one or more internal fixators. The one or more internal fixators include fixators that are inserted into and secured within the IM canal, such as deployable “talons.” These internal fixators may be positioned at a distal end of the IM nail (opposite the insertion location of the IM nail into the fractured bone). Thus, an actuator is often advanced along an internal lumen of the IM nail, along a length of the IM nail, to engage and deploy the internal fixators. Due to size constraints within the IM canal of many bones (e.g., a fibula), a permanent bone fixator (e.g., a bone screw) inserted into a fixator receiving hole of the IM nail blocks a path that the actuator advances along toward the internal fixators.

Accordingly, some embodiments described herein provide provisional fixation of the IM nail (e.g., via the temporary fixators) to one bone fragment (e.g., a distal fragment) without blocking a path of an actuator that advances along a length of the IM nail to engage and deploy the internal fixators. The internal fixators may be talons that contact inner walls of another fragment (e.g., a proximal fragment) of the fractured bone to secure the IM nail to the previously unsecured fragment of the fracture bone.

Some embodiments of the multi-lumen cannula described herein may be reversibly receivable within a recess of a targeting guide. The multi-lumen cannula being reversibly receivable may enable consistent placement of the temporary fixators while providing multiple insertion options for the permanent fixator. For example, the multi-lumen cannula may be receivable in the recess in a first orientation to achieve a static fixation of the IM nail to the fractured bone, and may further be receivable in the recess in a second orientation to apply compression to the fixation of the IM nail to the fractured bone.

The position of the first lumen and the second lumen (that receive the temporary fixators) may be consistent relative to the targeting guide in both the first orientation and the second orientation. The position of the third lumen (that receives the permanent fixator) changes relative to the targeting guide from the first orientation to the second orientation. For example, in the first orientation the third lumen may be closer to the fracture site, and in the second orientation the third lumen may be farther from the fracture site, or vice versa.

Some embodiments of a method of using of a multi-lumen cannula described herein comprises inserting a distal end of a multi-lumen cannula through a recess of a targeting guide. The method further comprises advancing the distal end toward an implant secured to the targeting guide, advancing a first bone fixator along a first linear axis of a first lumen extending through a body of the multi-lumen cannula, and advancing a second bone fixator along a second linear axis of a second lumen extending through the body of the multi-lumen cannula. The method further includes bracketing the implant between the first bone fixator and the second bone fixator with respect to a direction perpendicular to the first linear axis. A third bone fixator is advanced along a third linear axis of a third lumen extending through the body of the multi-lumen cannula according to the method, and the third bone fixator is advanced into a through hole of the implant.

Some embodiments of the method may include positioning the implant in an intramedullary canal of a bone such that a first portion of the implant is in a proximal fragment of the bone and a second portion of the implant is in a distal fragment of the bone. The proximal fragment is separated from the distal fragment by a fracture. The method may include advancing the first bone fixator along the first linear axis of the first lumen and into the distal fragment, and advancing the second bone fixator along the second linear axis of the second lumen and into the distal fragment. A force may be applied to the multi-lumen cannula, the targeting guide, the first bone fixator, the second bone fixator, or any combination thereof as part of the method, thereby moving the distal fragment away from the proximal fragment.

Some embodiments of a multi-lumen cannula described herein comprise a proximal end, a distal end, and a body extending therebetween such that the body is symmetrical about a first plane. The multi-lumen cannula further comprises a first lumen extending through the body along a first lumen axis parallel to and offset from the first plane and a second lumen extending through the body along a second lumen axis parallel to the first lumen axis and offset from the first plane. A third lumen of the multi-lumen cannula extends through the body along a third lumen axis parallel to the first lumen axis and positioned within the first plane. The first lumen axis and the second lumen axis are positioned within a second plane perpendicular to the first plane.

Some embodiments of a multi-lumen cannula described herein comprise a proximal end, a distal end, and a body extending therebetween. A first lumen of the multi-lumen cannula extends through the body from the proximal end to the distal end along a first lumen axis positioned within a first plane. A second lumen of the multi-lumen cannula extends through the body from the proximal end to the distal end along a second lumen axis. The second lumen axis is parallel to the first lumen axis and is positioned within the first plane offset from the first lumen axis in a first direction that is perpendicular to the first lumen axis. A third lumen of the multi-lumen cannula extends through the body from the proximal end to the distal end along a third lumen axis that is parallel to the first lumen axis. The third lumen axis is positioned within a second plane that is parallel to the first plane and offset from the first plane in a second direction that is perpendicular to the first plane. The first lumen and the second lumen each are positioned between the second plane and a third plane that is parallel to the second plane and tangent to the third lumen.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings. The headings and Abstract of the Disclosure provided herein are for convenience only and do not interpret the scope or meaning of the embodiments.

FIG. 1 is an anterior view of a fractured fibula.

FIG. 2 is an anterior view of the fractured fibula illustrated in FIG. 1 with the fractured fibula being prepared to receive an intramedullary implant.

FIG. 3 is an anterior view of the fractured fibula illustrated in FIG. 1 with the intramedullary implant being inserted into an intramedullary canal of the fractured fibula and a targeting guide attached to the intramedullary implant.

FIG. 4 is a front, top, isometric view of a multi-lumen cannula.

FIG. 5 is a rear, bottom, isometric view of the multi-lumen cannula illustrated in FIG. 4.

FIG. 6 is a front, elevation view of the multi-lumen cannula illustrated in FIG. 4 in a first orientation.

FIG. 7 is a front, elevation view of the multi-lumen cannula illustrated in FIG. 4 in a second orientation.

FIG. 8 is a front, elevation view of the multi-lumen cannula illustrated in FIG. 4 with another arrangement of lumens.

FIG. 9 is a side, elevation view of the multi-lumen cannula illustrated in FIG. 4.

FIG. 10 is a top, plan view of the multi-lumen cannula illustrated in FIG. 4.

FIG. 11 is a lateral view of the fractured fibula, intramedullary implant, and targeting guide illustrated in FIG. 3.

FIG. 12 is a lateral view of the fractured fibula, intramedullary implant, and targeting guide illustrated in FIG. 11, with the multi-lumen cannula in a first orientation.

FIG. 13 is a lateral view of the fractured fibula, intramedullary implant, and targeting guide illustrated in FIG. 12, with the multi-lumen cannula in a second orientation.

FIG. 14 is a posterior-lateral view of the fractured fibula, intramedullary implant, targeting guide, and multi-lumen cannula illustrated in FIG. 13, with temporary bone fixators inserted into a fragment of the fractured fibula.

FIG. 15 is a medial view of the fractured fibula, intramedullary implant, targeting guide, and temporary bone fixators illustrated in FIG. 14.

FIG. 16 is an anterior view of the fractured fibula, intramedullary implant, targeting guide, multi-lumen cannula, and temporary bone fixators illustrated in FIG. 14, with a force being applied to the fragment of the fractured bone.

FIG. 17 is an anterior view of the fractured fibula, intramedullary implant, targeting guide, multi-lumen cannula, and temporary bone fixators illustrated in FIG. 16, with an actuator deploying internal fixators of the intramedullary implant.

FIG. 18 is an anterior view of the fractured fibula, intramedullary implant, targeting guide, multi-lumen cannula, and temporary bone fixators illustrated in FIG. 17, with a permanent bone fixator securing the intramedullary implant to the fragment of the fractured bone.

FIG. 19 is an anterior view of the fractured fibula and intramedullary implant illustrated in FIG. 18, with permanent bone fixators securing the intramedullary implant to the fragment of the fractured bone and an end cap being secured to the intramedullary implant.

FIG. 20 is an anterior view of the intramedullary implant illustrated in FIG. 19 secured within the fractured fibula upon completion of a surgical procedure to repair the fractured fibula.

### DETAILED DESCRIPTION

As noted above, conventional targeting guides are known and utilized to secure hardware, such as intramedullary nails, to bone. Some known intramedullary implants push together fragments of a fractured bone separated by a fracture. Securing the fragments of the fractured bone when pushed together may result in a shortening of the fractured bone (e.g., compared to its pre-fractured condition and/or compared to a corresponding bone on the other side of the body, such as a fibula).

Some embodiments of the multi-lumen cannula described herein advantageously enable temporary bone fixators to be advanced into a first fragment of the fractured bone such that the temporary bone fixators are supported along a portion (e.g., at least a majority) of their length between a targeting guide that receives the multi-lumen cannula and the fractured bone. With the length of the temporary bone fixators supported, a force can be applied to the first fragment of the fractured bone (e.g., via the targeting guide and the supported temporary bone fixators) to move the first fragment relative to a second fragment of the fractured bone so as to achieve a desired length and/or a desired orientation.

In some cases, the multi-lumen cannula defines respective paths for the temporary bone fixators to pass on either side of (e.g., bracket) the intramedullary implant, without passing through the intramedullary implant. Thus, the multi-lumen cannula may be used as a fracture reduction sleeve that advantageously provisionally secures the intramedullary implant relative to the first bone fragment without blocking a path through the intramedullary implant along which an actuator travels to engage and deploy an internal fixator of the intramedullary implant. The internal fixator may be positioned within the second fragment of the fractured bone such that upon deployment of the internal fixator the internal fixator engages an inner wall of the second fragment thereby securing the intramedullary implant to the second fragment.

Conventional bone fixation systems include guides that provide provisional fixation via a temporary bone fixator that passes through or along one side of an intramedullary implant. As described above, passage through the intramedullary implant blocks an actuator from reaching a far end of the intramedullary implant to deploy internal bone fixators. Unbalanced temporary bone fixators (e.g., on one side of the intramedullary implant) may result in twisting or rotational forces being applied to the first bone fragment, which may, detrimentally, move the first bone fragment out of alignment with the second bone fragment.

Some known bone fixation systems provide a curved path for the temporary bone fixator to avoid contact with the intramedullary implant. However, the curved path may result in less stable fixation. Advantageously, the linear lumens of some embodiments of the disclosed multi-lumen cannula enable the temporary bone fixators to be advanced into the first bone fragment, and then subsequently advanced again (e.g., further into the first bone fragment and/or into a second bone (e.g., different than the fractured bone) to provisionally secure the first bone fragment to the second bone until permanent bone fixators are inserted.

Depending on the specific bone fractured, and the location of the fracture within the specific bone, one or more of the permanent fixators may have options for their placement through the intramedullary implant. For example, a static fixation may be achieved by insertion of a bone screw through a first portion of a bone fixation hole in the intramedullary implant, and a compression fixation may be achieved by insertion of the bone screw through a second portion of the bone fixation hole.

Advantageously, some embodiments of the multi-lumen cannula may be symmetrical about a plane of symmetry. The multi-lumen cannula may be insertable into a recess of the targeting guide in two different orientations. The locations of the first lumen and the second lumen (and the resulting location of the temporary bone fixator inserted therethrough) relative to the first fragment of the fractured bone may be the same in both of the two orientations. For example, the first lumen and the second lumen may swap positions when transitioning from the first orientation to the second orientation, such that the two temporary bone fixators collectively are in the same location.

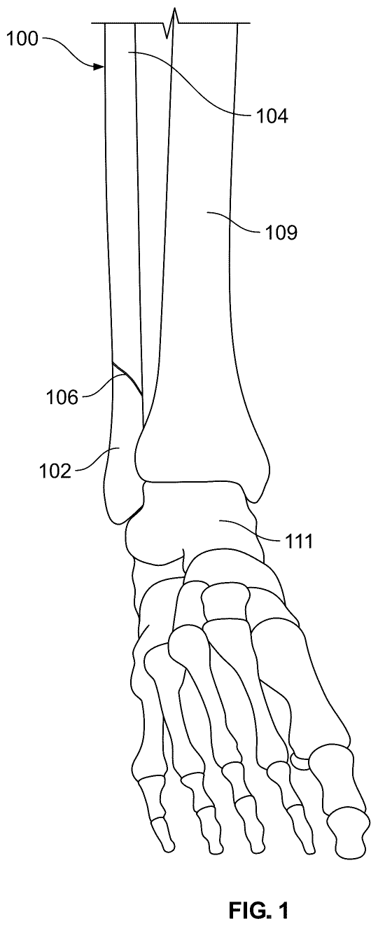

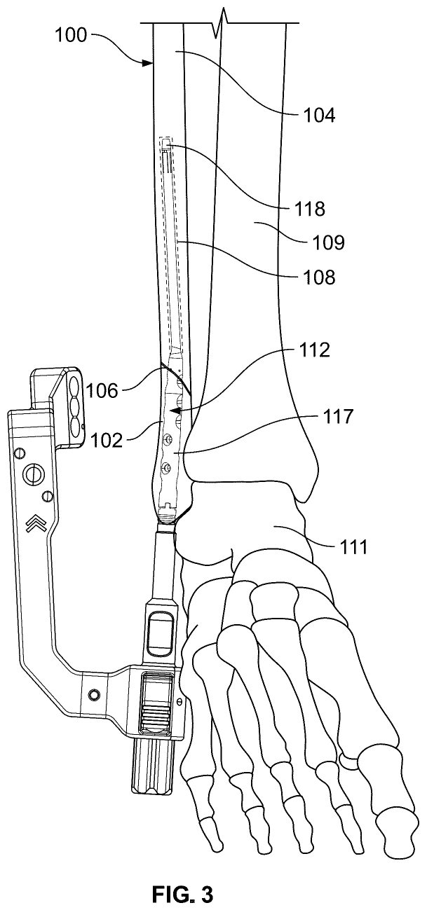

Referring now to the drawings, and specifically to FIGS. 1 to 3, a fractured bone 100 may comprise two or more fragments (e.g., a first fragment 102 and a second fragment 104) separated by one or more fractures 106. For example, the fractured bone 100 may be a fibula, the first fragment 102 may be a distal fragment, and the second fragment 104 may be a proximal fragment. As shown in FIG. 1, prior to insertion of any fixation hardware, the first fragment 102 and the second fragment 104 may be aligned (e.g., to approximate the shape of the fractured bone 100 prior to the fracture 106—referred to herein as the “pre-fracture condition”). A clamp (not shown) may be used to hold the first fragment 102 and the second fragment 104 in place.

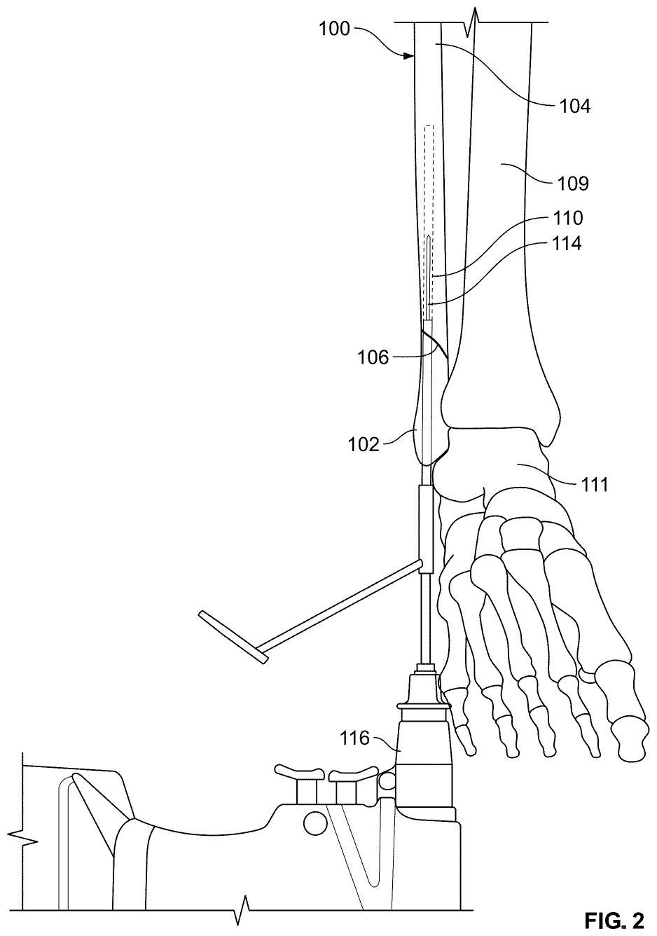

As shown in FIG. 2, instruments may be used to form a hole 108 within the fractured bone 100. The hole 108 may include an intramedullary canal 110 of the fractured bone 100. The intramedullary canal 110 may be enlarged along at least a portion of the length of the fractured bone 100 to a size that can accommodate an intramedullary implant 112. For example, a guide wire 114 may be inserted through a surface in the first fragment 102 (e.g., a distal surface of the lateral malleolus) and into the intramedullary canal 110. The guide wire 114 may be advanced (e.g., proximally/anteriorly) across the fracture 106 into the intramedullary canal 110 of the second fragment 104. A drill or reamer 116 may be used to form the hole 108 (e.g., by enlarging the intramedullary canal 110 to a desired size). The drill or reamer 116 may be cannulated and inserted over the guide wire 114 and advanced along the guide wire 114 through the first fragment 102, across the fracture 106, and into the second fragment 104.

Once the hole 108 is formed, the intramedullary implant 112 (e.g., an intramedullary nail 117) may be positioned within the hole 108. For example, a tip 118 of the intramedullary nail 117 may be advanced (e.g., proximally/anteriorly) through a surface in the first fragment 102 (e.g., the distal surface of the lateral malleolus), across the fracture 106, and into the second fragment 104. Once positioned in the hole 108 as desired, the intramedullary nail 117 may be secured to both the first fragment 102 and the second fragment 104 of the fractured bone 100, as described in further detail below.

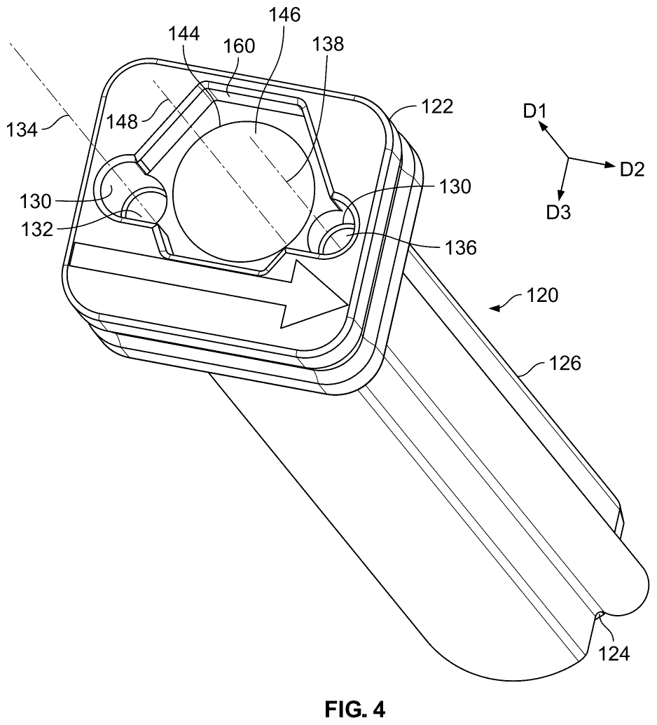

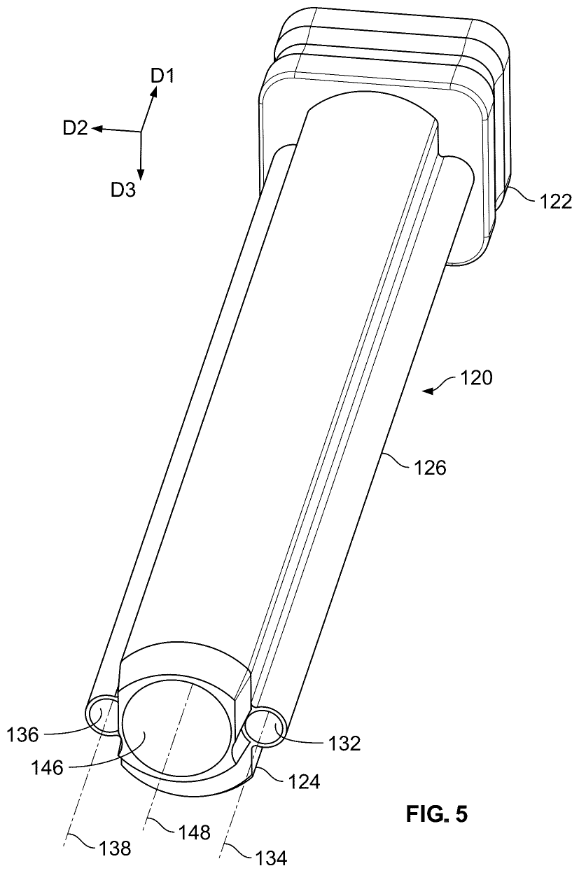

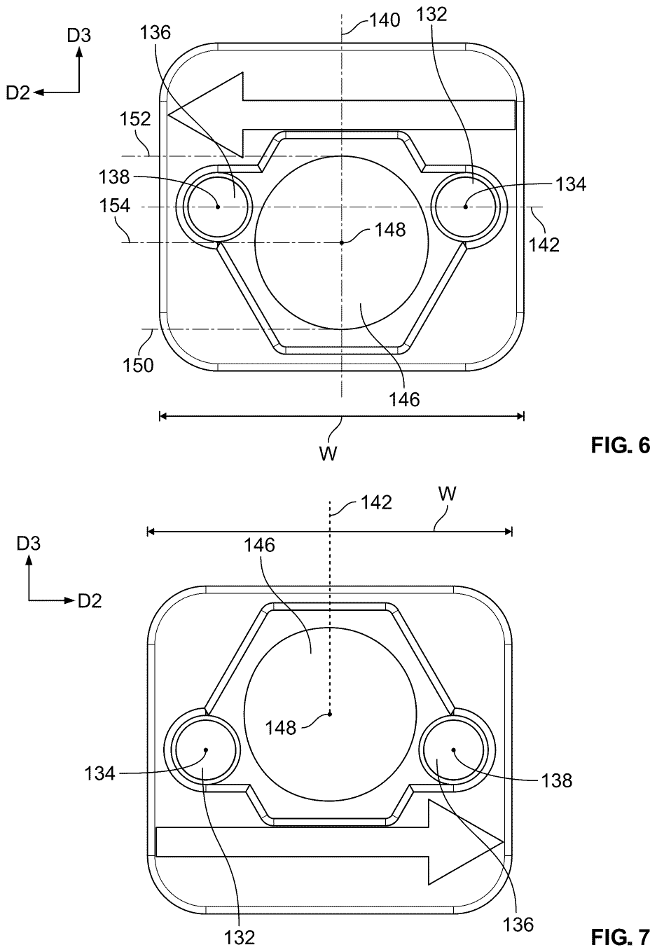

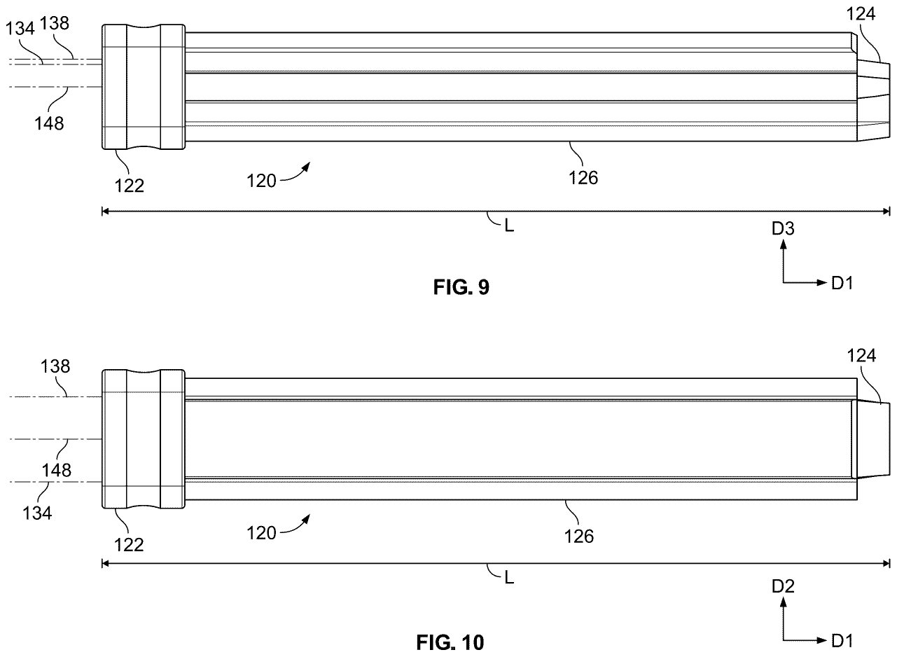

Referring to FIGS. 4 to 10, a multi-lumen cannula 120 may comprise a proximal end 122, a distal end 124, and a body 126 extending therebetween. The multi-lumen cannula 120 may define a length L along which the body 126 extends from the proximal end 122 to the distal end 124. As shown, the body 126 may be elongate along the length L. The multi-lumen cannula 120 may comprise one or more temporary bone fixation lumens 130, each extending through the body 126 along respective axes. For example, the temporary bone fixation lumens 130 may include a first lumen 132 extending through the body 126 along a first lumen axis 134, and a second lumen 136 extending through the body 126 along a second lumen axis 138.

The first lumen axis 134 and the second lumen axis 138 may each be central axes of the respective lumen. The first lumen axis 134 and the second lumen axis 138 may be parallel to each other. As shown, the first lumen axis 134 and the second lumen axis 138 may each be parallel to a first direction D1, along which the length L of the multi-lumen cannula 120 is measured. The temporary bone fixation lumens 130 may each be sized to receive a temporary bone fixator (e.g., a K-wire).

Some embodiments of the body 126 may be symmetrical (e.g., about a plane of symmetry 140). The body 126 may include two equal portions separated by the plane of symmetry 140, which may be parallel to the first lumen axis 134, the first direction D1, the length L, or any combination thereof. As described herein, the body 126 being symmetrical may advantageously enable the multi-lumen cannula 120 to establish multiple insertion paths for a permanent bone fixator (e.g., a bone screw) through a fastener receiving hole of the intramedullary implant 112. However, it should be understood that embodiments of the multi-lumen cannula 120 may be symmetrical about multiple planes of symmetry, or not symmetrical about any planes of symmetry.

As shown in the illustrated embodiment, the first lumen axis 134 may be parallel to and offset from the plane of symmetry 140 (e.g., in a second direction D2 that is perpendicular to the first direction D1). The multi-lumen cannula 120 may define a width W along which the body 126 extends in the second direction D2. The second lumen axis 138 may also be parallel to and offset from the plane of symmetry 140 (e.g., in the second direction D2) such that the first lumen axis 134 is on one side of the plane of symmetry 140 and the second lumen axis 138 is on the other side of the plane of symmetry 140. According to some embodiments, the first lumen axis 134 and the second lumen axis 138 may be positioned in a second plane 142 that is perpendicular to the plane of symmetry 140.

The multi-lumen cannula 120 may comprise one or more permanent bone fixation lumens 144, each extending through the body 126 along respective axes. For example, the one or more permanent bone fixation lumens 144 may include a third lumen 146 extending through the body 126 along a third lumen axis 148. The third lumen axis 148 may be a central axis of the third lumen 146. The third lumen axis 148 may be parallel to one or both of the first lumen axis 134 and the second lumen axis 138. As shown, the third lumen axis 148 may be parallel to the first direction D1. The one or more permanent bone fixation lumens 144 may each be sized to receive a permanent bone fixator (e.g., a bone screw).

As shown in the illustrated embodiment, the third lumen axis 148 may be positioned within the plane of symmetry 140 (e.g., such that the third lumen 146 has equal portions on either side of the plane of symmetry 140). The third lumen axis 148 may also be parallel to and offset from the second plane 142 (e.g., in a third direction D3 that is perpendicular to both the first direction D1 and the second direction D2). As shown the second plane 142 may intersect the third lumen 146. According to some embodiments, the third lumen axis 148 may be positioned within both the plane of symmetry 140 and the second plane 142.

According to some embodiments, the one or more temporary bone fixation lumens 130 and the one or more permanent bone fixation lumens 144 (e.g., the first lumen 132, the second lumen 136, and the third lumen 146) may extend through an entirety of the length L of the body 126, from the proximal end 122 to the distal end 124. One or more of the lumens may have different lengths (e.g., the length L of a portion of the body 126 that defines the third lumen 146 may be greater than the length L of another portion of the body 126 that defines the first lumen 132).

The third lumen 146 may be positioned within the body 126 between a third plane 150 and a fourth plane 152. As shown, the third plane 150 and the fourth plane 152 may each be tangent to the third lumen 146 (such that the third lumen is bracketed by the third plane 150 and the fourth plane 152) and parallel to the second plane 142. The first lumen 132 and the second lumen 136 may be positioned between the third plane 150 and the fourth plane 152 (e.g., with respect to the third direction D3). According to some embodiments, the third lumen 146 may be positioned within the body 126 such that the third lumen axis 148 is positioned within a fifth plane 154 that is parallel to the second plane 142. The first lumen 132 and the second lumen 136 may be positioned between the fifth plane 154 and the fourth plane 152 (e.g., with respect to the third direction D3).

The lumens of the multi-lumen cannula 120 may each have a respective cross-sectional shape defined within a plane 162 perpendicular to the respective axis of each of the lumens. For example, the first lumen 132 may be cylindrical and the cross-sectional shape of the first lumen 132 may be a circle. Many temporary and permanent bone fixators have a circular cross-sectional shape, and the shape of the lumens of the multi-lumen cannula 120 may be selected to match the shape of the fixator to be inserted therethrough (or vice versa). However, the lumens are not limited to any particular cross-sectional shape (circular or otherwise).

The lumens of the multi-lumen cannula 120 may be different sizes. For example, the one or more temporary bone fixation lumens 130 may be smaller than the one or more permanent bone fixation lumens 144. According to one embodiment, the cross-sectional shape of the first lumen 132 may be substantially the same as (e.g., identical to) the cross-sectional shape of the second lumen 136. The lumens of the multi-lumen cannula 120 may define a maximum cross-sectional dimension (e.g., a diameter for a lumen with a circular cross-sectional shape). The maximum cross-sectional dimension of the third lumen 146 may be greater than the maximum cross-sectional dimension of the first lumen 132 and/or the second lumen 136.

For example, the one or more temporary bone fixation lumens 130 may be sized to receive a K-wire (e.g., with a maximum cross-sectional dimension of about 2 mm) and the one or more permanent bone fixation lumens 144 may be sized to receive a bone screw (e.g., with a maximum cross-sectional dimension of about 5 mm). According to some embodiments, the one or more temporary bone fixation lumens 130 may have a maximum cross-sectional dimension between about 1 and about 5 times a maximum cross-sectional dimension of the one or more permanent bone fixation lumens 144. The first lumen 132 may define a length measured along the first lumen axis 134 and the length of the first lumen axis 134 may be between 10 and 100 times greater than a maximum cross-sectional dimension J1 of the first lumen 132.

The length of the first lumen 132 may range from about 50 mm to about 500 mm. In some cases, the lengths of the first lumen 132 may range from 100 mm to 400 mm, e.g., from 150 mm to 300 mm, or from 200 mm to 250 mm. In terms of lower limits, the length of the first lumen 132 may be greater than 50 mm, e.g., greater than 100 mm, greater than 150 mm, greater than 200 mm, greater than 200 mm, greater than 300 mm, or greater than 400 mm. In terms of upper limits, the length of the first lumen 132 may be less than 500 mm, e.g., less than 400 mm, less than 300 mm, less than 250 mm, less than 200 mm, less than 150 mm, or less than 100 mm.

The maximum cross-sectional dimension J1 of the first lumen 132 may range from about 0.5 mm to about 5.0 mm. In some cases, the maximum cross-sectional dimension J1 of the first lumen 132 may range from 1.0 mm to 4.0 mm, e.g., from 1.5 mm to 3.0 mm, or from 2.0 mm to 2.5 mm. In terms of lower limits, the maximum cross-sectional dimension J1 of the first lumen 132 may be greater than 0.5 mm, e.g., greater than 1.0 mm, greater than 1.5 mm, greater than 2.0 mm, greater than 2.5 mm, greater than 3.0 mm, or greater than 4.0 mm. In terms of upper limits, the maximum cross-sectional dimension J1 of the first lumen 132 may be less than 5.0 mm, e.g., less than 4.0 mm, less than 3.0 mm, less than 2.5 mm, less than 2.0 mm, less than 1.0 mm, or less than 1.0 mm.

Some embodiments of the body 126 may have a cross-sectional shape with an outer perimeter that is “keyed” along at least a portion of the length L so as to be non-rotatably receivable within a corresponding recess of a targeting guide. For example, the body 126 may have a cross-sectional shape with an outer perimeter that is non-circular along at least a portion of the length L.

The multi-lumen cannula 120 may include a recess 160 extending into the body 126 from the proximal end 122 toward the distal end 124. The recess 160 may be positioned such that the first lumen axis 134, the second lumen axis 138, the third lumen axis 148, or any combination thereof intersects the recess 160. According to some embodiments, a cross-sectional shape of the recess 160 may be non-circular. For example, the cross-sectional shape of the recess 160 may be a polygon with one or more flat edges. The cross-sectional shape may be selected such that the recess 160 receives a corresponding tool while preventing rotation of the tool relative to the recess 160.

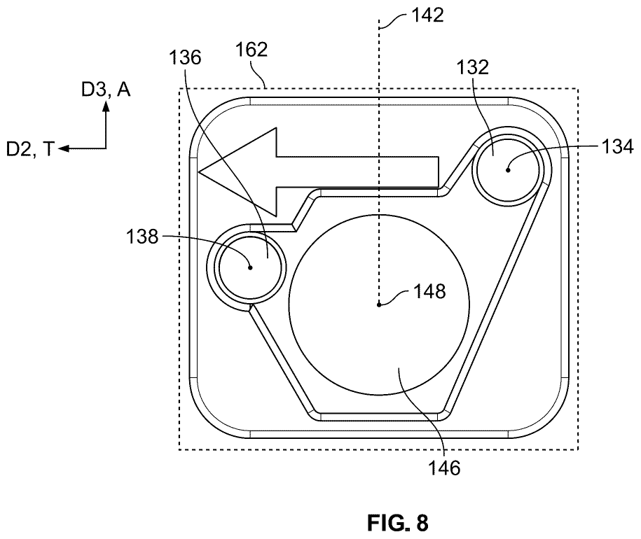

As shown in FIG. 8, the first linear axis 134 may be offset from the second linear axis 138 in both a lateral direction A (e.g., the third direction D3) and a transverse direction T (e.g., the second direction D2). Thus, according to some embodiments, both the second linear axis 138 and the third linear axis 148 may be offset from the first linear axis 134 in two perpendicular directions (e.g., the second direction D2 and the third direction D3). For such an embodiment, the multi-lumen cannula 120 may be devoid of the plane of symmetry 140 (e.g., may be asymmetrical). The first lumen 132 and the second lumen 136 may have matching cross-sectional shapes defined within a plane (e.g., the plane 162) perpendicular to the first lumen axis 134. The third lumen 146 may have a cross-sectional shape that is different from the cross-sectional shapes of the first lumen 132 and the second lumen 136. As shown, the third lumen axis 148 may be positioned within the plane 142 that is parallel to the lateral direction A, and the first lumen axis 134 and the second lumen axis 138 may be positioned equidistant from the plane 142 with respect to the transverse direction T.

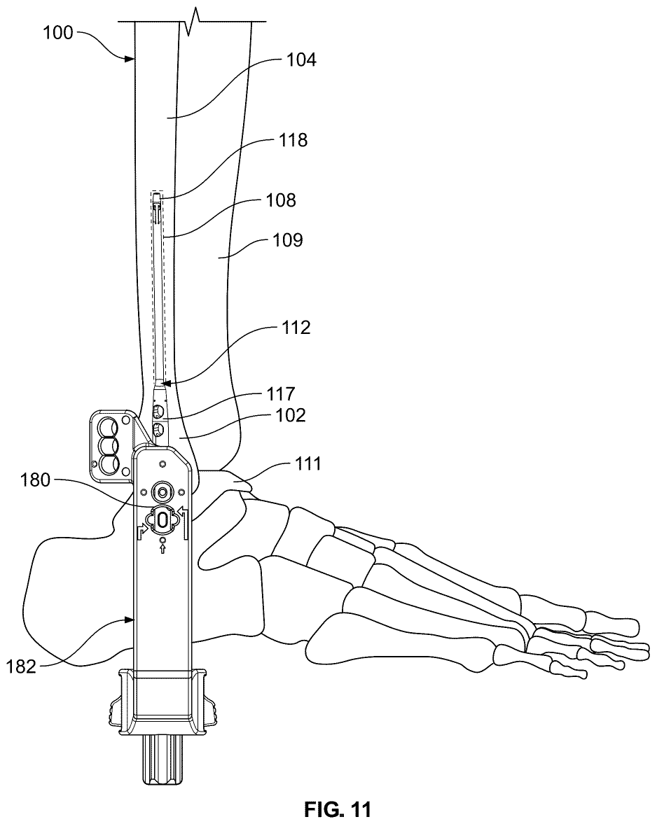

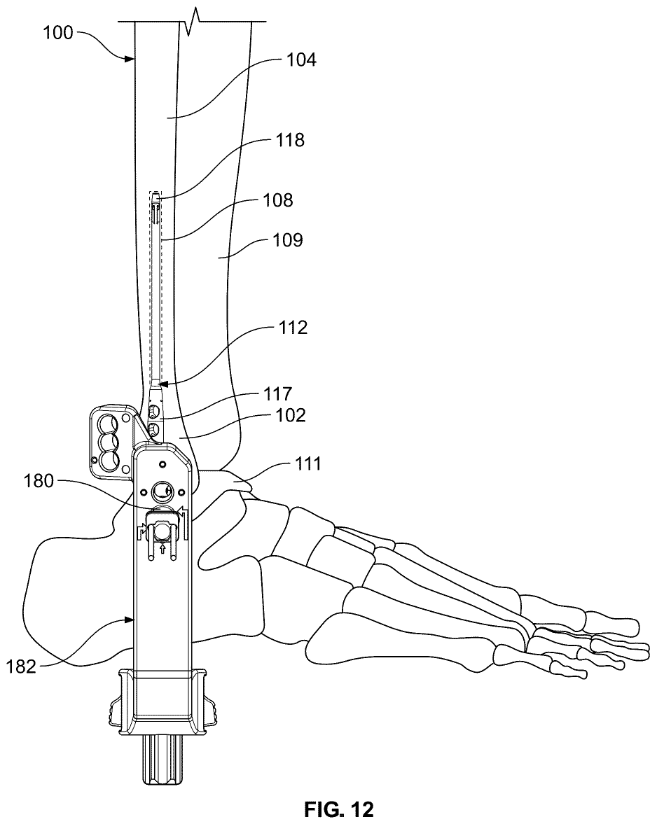

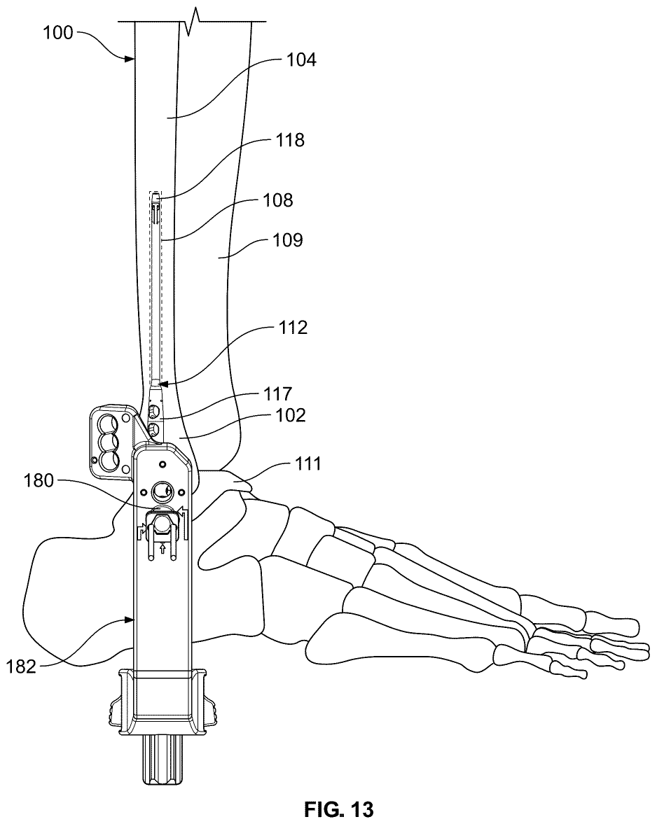

Referring to FIGS. 11 to 20, a method of using of the multi-lumen cannula 120 may be performed during testing and assembly of a bone fracture repair system. As shown in FIGS. 12 and 13, the method may comprise inserting the distal end 124 of the multi-lumen cannula 120 through a recess 180 of a targeting guide 182 that is secured to the intramedullary nail 117. The recess 180 may have a “keyed” shape that corresponds to the outer perimeter of the cross-sectional shape of the body 126 of the multi-lumen cannula 120. Thus, when positioned within the recess 180, the multi-lumen cannula 120 may not be rotatable relative to the targeting guide 182. The method may further include advancing the distal end 124 toward the intramedullary nail 117 (e.g., along the first direction D1). The distal end 124 may be advanced toward the intramedullary nail 117 until a gap between the multi-lumen cannula 120 and the intramedullary nail 117 is minimized (e.g., below about 20 mm).

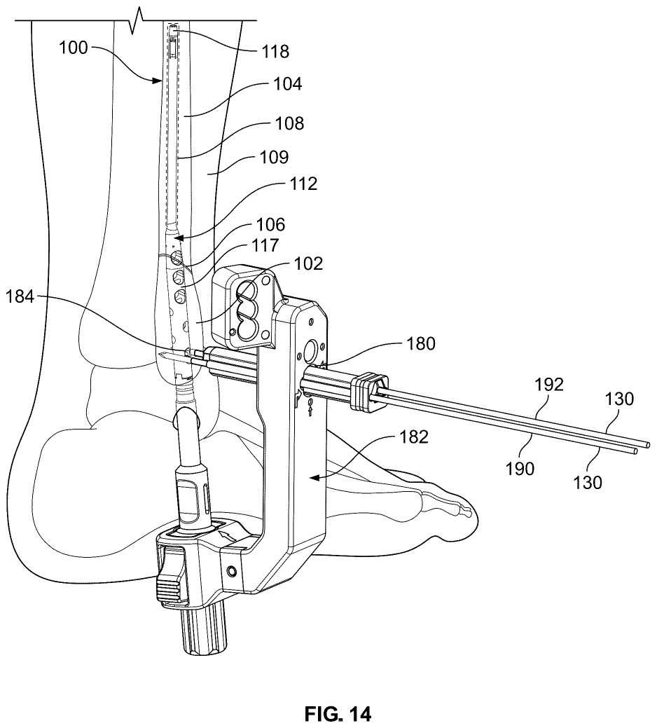

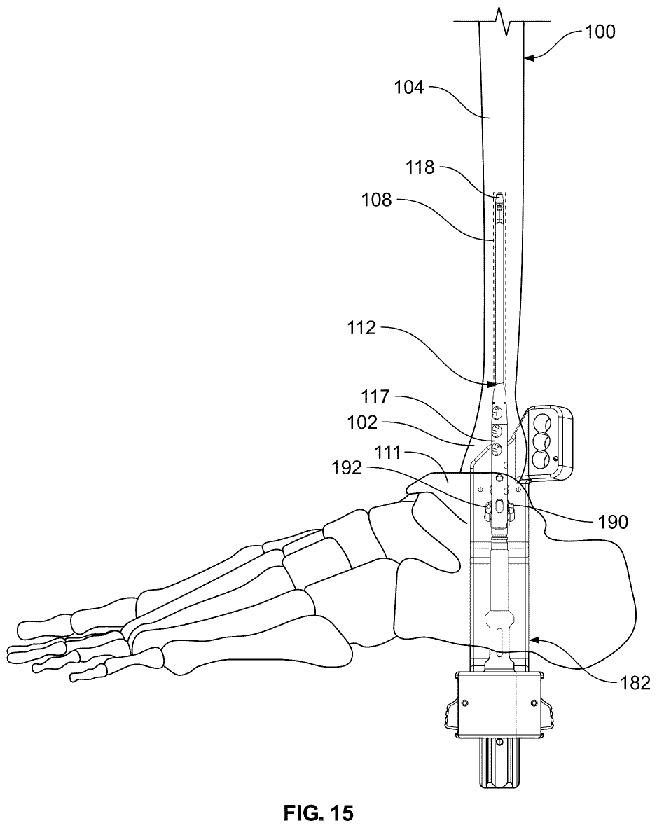

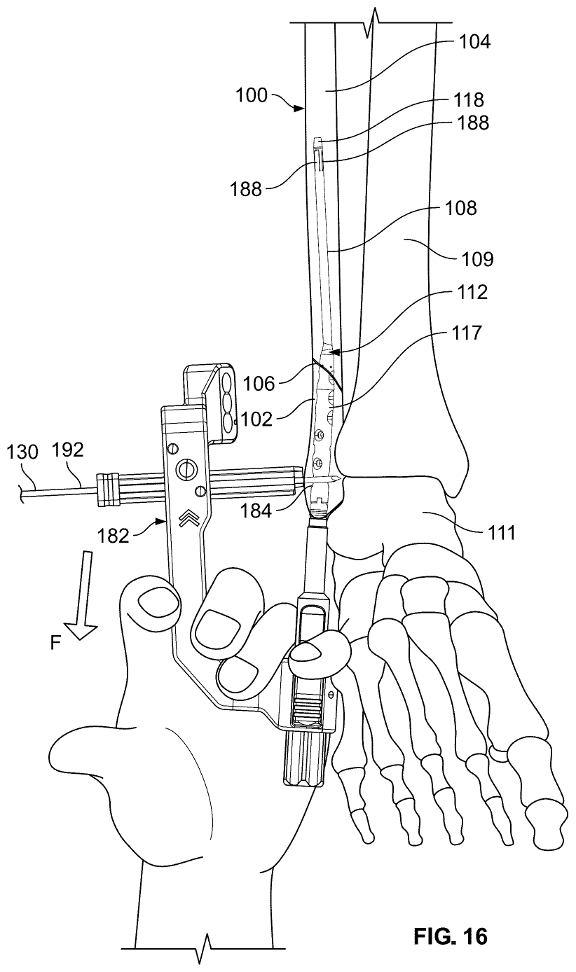

As shown in FIG. 14, the method may further include advancing a first bone fixator 190 (e.g., a K-wire) through the first lumen 132 (e.g., along the first lumen axis 134). Similarly, the method may further include advancing a second bone fixator 192 (e.g., a K-wire) through the second lumen 136 (e.g., along the second lumen axis 138). When the multi-lumen cannula 120 is positioned within the recess 180 and the targeting guide 182 is secured to the intramedullary nail 117, the first bone fixator 190 may be advanced along the first lumen axis 134 passing on one side of the intramedullary nail 117, and the second bone fixator 192 may be advanced along the second lumen axis 138 passing on the other side of the intramedullary nail 117. As shown in FIG. 15, the method may include bracketing the intramedullary nail 117 between the first bone fixator 190 and the second bone fixator 192 with respect to the second direction D2.

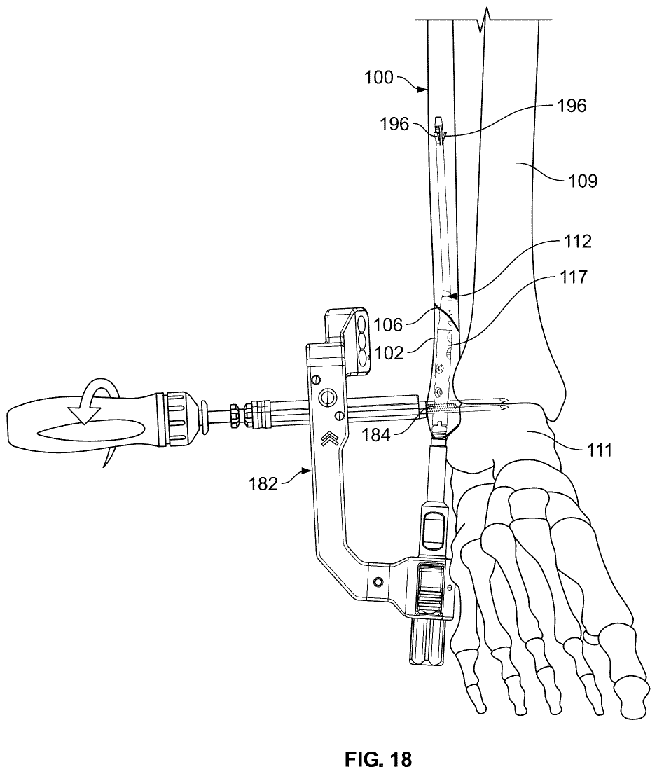

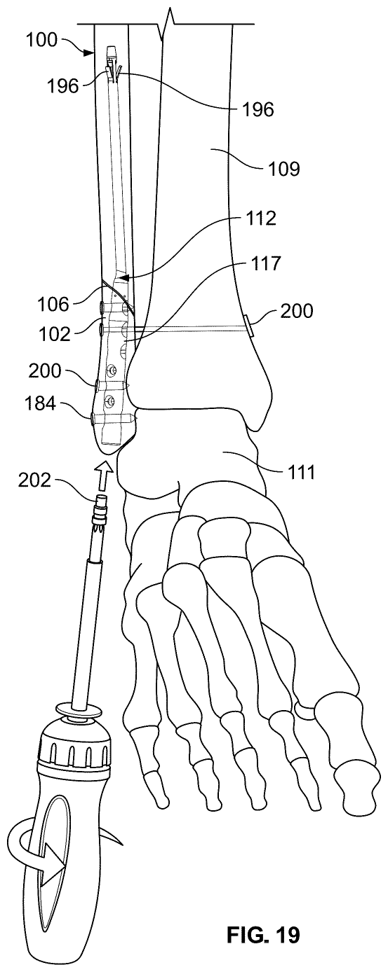

As shown in FIG. 18, the method may further include advancing a third bone fixator 194 (e.g., a bone screw) through the third lumen 146 (e.g., along the third lumen axis 148). When the multi-lumen cannula 120 is positioned within the recess 180 and the targeting guide 182 is secured to the intramedullary nail 117, the third bone fixator 194 may be advanced along the third lumen axis 148 until passing through a bone fastener receiving hole 184 of the intramedullary nail 117. According to some embodiments, the third lumen axis 148 and the intramedullary nail 117 may be substantially perpendicular to one another (e.g., within about 10 degrees of offset). Thus, a central axis of the bone fastener receiving hole 184 may be perpendicular to the third lumen axis 148, or offset from the third lumen axis 148 by up to about 10 degrees.

The multi-lumen cannula 120 may be in a first orientation relative to the targeting guide 182 when the distal end 124 of the multi-lumen cannula 120 is inserted through the recess 180, as shown in FIG. 12. The method may further comprise withdrawing the distal end 124 of the multi-lumen cannula 120 through the recess 180 of the targeting guide 182 (e.g., opposite the direction along which the distal end 124 was inserted into the recess 180). The multi-lumen cannula 120 may be rotated (e.g., 180 degrees about the first lumen axis 134) to a second orientation, and then the distal end 124 may be inserted through the recess 180 of the targeting guide 182 while the multi-lumen cannula 120 is in the second orientation. When the multi-lumen cannula 120 is positioned within the recess 180 in the second orientation, the first bone fixator 190, the second bone fixator 192, and/or the third bone fixator 194 may be inserted through the first lumen 132, the second lumen 136, and/or the third lumen, respectively, as described above in reference to the first orientation.

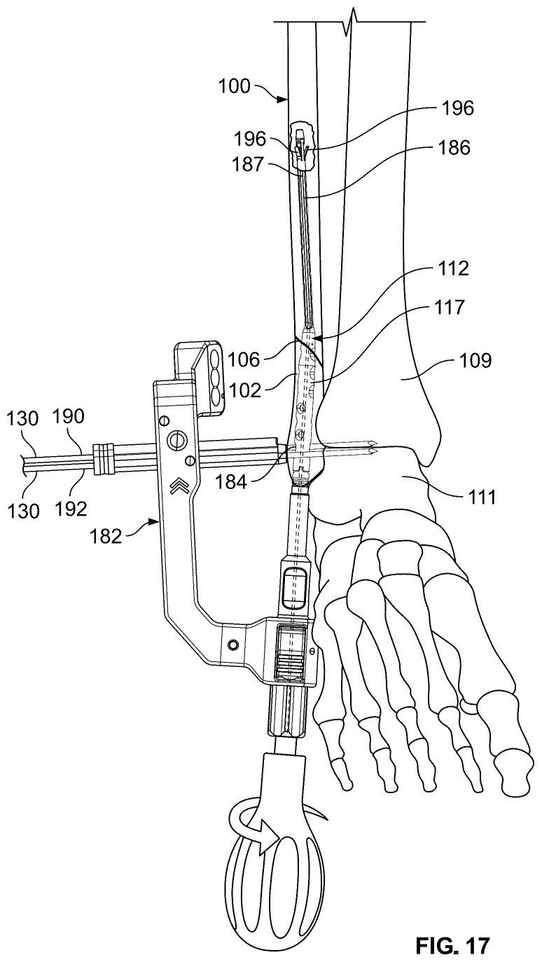

As shown in FIG. 17, the method may include advancing an actuator 186 along a length of the intramedullary nail 117, passing the actuator 186 between the first bone fixator 190 and the second bone fixator 192. According to some embodiments of the method, the actuator 186 may be advanced prior to advancing the third bone fixator 194 into the bone fastener receiving hole 184 of the intramedullary nail 117. The actuator 186 may be advanced through a channel within the intramedullary nail 117, and the channel may be blocked by the third bone fixator 194, when the third bone fixator 194 is positioned within the bone fastener receiving hole 184.

The method may include advancing an actuator 186 within the intramedullary nail 117 until the actuator 186 engages a deployable bone fixator 196 of the intramedullary nail 117. The actuator 186 may be rotated within the intramedullary nail 117 while the actuator 186 is engaged with the deployable bone fixator 196 to deploy the deployable bone fixator 196 (e.g., such that the deployable bone fixator 196 engages with an inner surface of the second fragment 104). For example, the deployable bone fixator 196 may include one or more talons 188 that are movable from a collapsed configuration (with a reduced cross-section) used during insertion of the intramedullary nail 117, to a deployed configuration (with an expanded cross-section). Once the deployable bone fixator 196 is in the deployed configuration, the method may include withdrawing the actuator 186 from the intramedullary nail 117 prior to advancing the third bone fixator 194 into the bone fastener receiving hole 184.

According to some embodiments, the method of using of the multi-lumen cannula 120 may be a method of repairing a fractured bone (e.g., the fractured bone 100). The method may include positioning the intramedullary nail 117 in an intramedullary canal 110 (e.g., the hole 108) of the fractured bone 100 such that a portion of the intramedullary nail 117 is in the first fragment 102 of the fractured bone 100, another portion of the intramedullary nail 117 is in the second fragment 104 of the fractured bone 100, and the intramedullary nail 117 extends across the fracture 106 between the first fragment 102 and the second fragment 104.

For example, when inserting the distal end 124 of the multi-lumen cannula 120 through the recess 180 of the targeting guide 182, the targeting guide 182 may be secured to the intramedullary nail 117, which may be positioned within the hole 108 formed in the fractured bone 100. The distal end 124 may be advanced toward the intramedullary nail 117, according to the method, until the distal end 124 contacts the skin of the patient or the fractured bone 100.

When the intramedullary nail 117 is positioned within the hole 108 formed in the fractured bone 100, the method may include advancing the first bone fixator 190 along the first lumen axis 134 and into the first fragment 102 (e.g., a distal fragment of a fibula) and advancing the second bone fixator 192 along the second lumen axis 138 and into the second fragment 104. As shown in FIG. 16, a force F may be applied to the multi-lumen cannula 120, the targeting guide 182, the first bone fixator 190, the second bone fixator 192, or any combination thereof. When the first bone fixator 190 is inserted into and secured to the first fragment 102 (e.g., a distal fragment of a fibula) and the second bone fixator 192 is inserted into and secured to the second fragment 104, the force F moves the first fragment 102 away from the second fragment 104. Thus, the method may include increasing a length of the fractured bone 100 with the multi-lumen cannula 120 used as a fracture reduction sleeve. The method may include rotating the first fragment 102 relative to the second fragment 104 of the fractured bone 100 (e.g., to realign the first fragment 102 and the second fragment 104 to match the pre-fractured condition of the fractured bone 100). However, the multi-lumen cannula 120 may be used in other procedures involving the insertion of bone fixators along desired pathways.

When the intramedullary nail 117 is positioned within the hole 108 formed in the fractured bone 100, the method may include securing the intramedullary nail 117 to the first fragment 102 via insertion of the third bone fixator 194. Transitioning the deployable bone fixator 196 may include abutting the one or more talons 188 with the inner surface of the second fragment 104 of the fractured bone 100. Advancing the actuator 186 along the length of the intramedullary nail 117 may include moving a tip 187 of the actuator 186 across the fracture 106 and passing the tip 187 between the first bone fixator 190 and the second bone fixator 192.

As shown in FIG. 16, when the fractured bone 100 has the desired length (e.g., while the force F is being applied to the first fragment 102 (e.g., via the multi-lumen cannula 120, the targeting guide 182, the first bone fixator 190, the second bone fixator 192, or any combination thereof) the first bone fixator 190 and the second bone fixator 192 may be advanced further (e.g., medially into the body) to enter a second bone (e.g., a tibia 109 and/or a talus 111). Thus the method may include securing a position of the first fragment 102 to another bone (e.g., not the fractured bone 100 or any of its fragments). When the first fragment 102 is secured to the other bone, the force F may be removed as it is no longer needed to maintain the desired length of the fractured bone 100, and the position of the first fragment 102 relative to the second fragment 104.

Rotating the actuator 186 within the intramedullary nail 117 may deploy the one or more talons 188 thereby securing the intramedullary nail 117 to the second fragment 104. After deploying the one or more talons 188, the actuator 186 may be withdrawn entirely from the intramedullary canal 110 of the intramedullary nail 117 prior to advancing the third bone fixator 194 into the bone fastener receiving hole 184, as shown in FIG. 18. Inserting the third bone fixator 194, according to the method, may secure the intramedullary nail 117 to the first fragment 102.

As part of the method, the first bone fixator 190 may be moved across a first distance between a distal end of the first lumen 132 and the first fragment 102 of the fractured bone 100, and the second bone fixator 192 may be moved across a second distance between a distal end of the second lumen 136 and the first fragment 102 of the fractured bone 100. The first distance and the second distance may each range between about 0 mm and 20 mm.

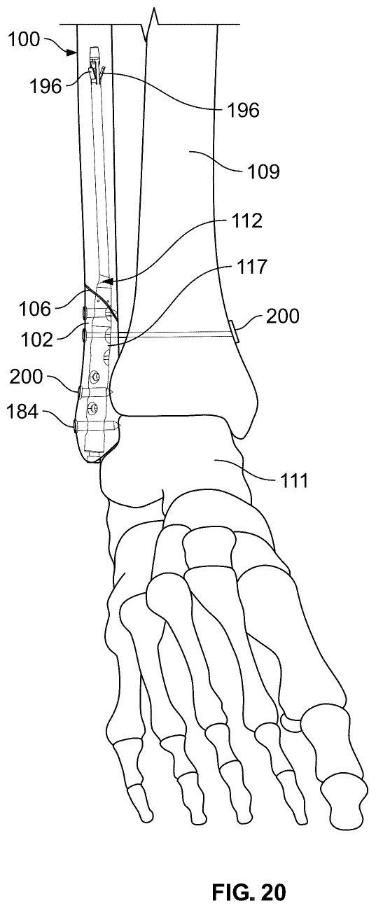

As shown in FIG. 19, additional permanent bone fixators 200 (e.g., bone screws similar to the third bone fixator 194) may be inserted into corresponding bone fastener receiving holes (e.g., similar to the bone fastener receiving hole 184) to secure the intramedullary nail 117 to the fractured bone 100. According to some embodiments, all of the additional permanent bone fixators 200 may be inserted into corresponding bone fastener receiving holes to secure the intramedullary nail 117 to the first fragment 102 of the fractured bone 100. After insertion of the additional permanent bone fixators 200 (e.g., when the intramedullary nail 117 is fully secured to the fractured bone 100), an end cap 202 may be coupled to the intramedullary nail 117 to close the path through which the actuator 186 passed. As shown in FIG. 20, the completed intramedullary nail 117 and the permanent bone fixators remain secured to the fractured bone 100, and the temporary bone fixators are removed.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The various embodiments described above can be combined to provide further embodiments.

Many of the methods described herein can be performed with variations. For example, many of the methods may include additional acts, omit some acts, and/or perform acts in a different order than as illustrated or described.

As used herein, “greater than” and “less than” limits may also include the number associated therewith. Stated another way, “greater than” and “less than” may be interpreted as “greater than or equal to” and “less than or equal to.” It is contemplated that this language may be subsequently modified in the claims to include “or equal to.” For example, “greater than 2 mm” may be interpreted as, and subsequently modified in the claims as “greater than or equal to 2 mm.”

In some embodiments, any or some of the components or steps disclosed herein may be considered optional. In some cases, the disclosed embodiments may expressly exclude any or some of the aforementioned elements or steps in this description, e.g., via claim language. For example, claim language may be modified to recite that the disclosed multi-lumen cannula and/or methods, etc., do not utilize or comprise a symmetrical body. Such negative limitations are contemplated, and this text serves as support for negative limitations for components, steps, and/or features.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of using a multi-lumen cannula, the method comprising:
inserting a distal end of a multi-lumen cannula through a recess of a targeting guide;
advancing the distal end toward an implant secured to the targeting guide;
advancing a first bone fixator along a first linear axis of a first lumen extending through a body of the multi-lumen cannula;
advancing a second bone fixator along a second linear axis of a second lumen extending through the body of the multi-lumen cannula;
bracketing the implant between the first bone fixator and the second bone fixator with respect to a direction perpendicular to the first linear axis;
advancing a third bone fixator along a third linear axis of a third lumen extending through the body of the multi-lumen cannula; and
advancing the third bone fixator into a through hole of the implant.

2. The method of claim 1 wherein the first linear axis and the second linear axis are positioned within a plane about which the body of the multi-lumen cannula is symmetrical.

3. The method of claim 2 wherein the third linear axis is offset from the plane in a direction perpendicular to both the first linear axis and the direction perpendicular to the first linear axis.

4. The method of claim 1 wherein the multi-lumen cannula is in a first orientation relative to the targeting guide when the distal end of the multi-lumen cannula is inserted through the recess, the method further comprising:
withdrawing the distal end of the multi-lumen cannula through the recess of the targeting guide;
rotating the multi-lumen cannula 180 degrees to a second orientation; and
inserting the distal end of the multi-lumen cannula through the recess of the targeting guide while the multi-lumen cannula is in the second orientation.

5. The method of claim 4, further comprising:
advancing the first bone fixator along the first linear axis of the first lumen while the multi-lumen cannula is in the second orientation and positioned within the recess;
advancing the second bone fixator along the second linear axis of the second lumen while the multi-lumen cannula is in the second orientation and positioned within the recess; and
bracketing the implant between the first bone fixator and the second bone fixator with respect to the direction perpendicular to the first linear axis while the multi-lumen cannula is in the second orientation and positioned within the recess.

6. The method of claim 1, further comprising:
advancing an actuator along a length of the implant between the first bone fixator and the second bone fixator prior to advancing the third bone fixator into the through hole of the implant;
rotating the actuator within the implant to deploy one or more internal fixators of the implant; and
withdrawing the actuator from the implant prior to advancing the third bone fixator into the through hole of the implant.

7. The method of claim 1, further comprising:
positioning the implant in an intramedullary canal of a fractured bone, wherein the fractured bone comprises a proximal fragment separated from a distal fragment by a fracture;
advancing the first bone fixator along the first linear axis of the first lumen and into the distal fragment;
advancing the second bone fixator along the second linear axis of the second lumen and into the distal fragment; and
applying a force to the multi-lumen cannula, the targeting guide, the first bone fixator, the second bone fixator, or any combination thereof thereby moving the distal fragment relative to the proximal fragment.

8. The method of claim 7, further comprising:
positioning a first portion of the implant in the intramedullary canal of the fractured bone such that a first portion of the implant is in the proximal fragment of the fractured bone and a second portion of the implant is in the distal fragment of the fractured bone, prior to applying the force.

9. The method of claim 8, further comprising:
advancing an actuator along a length of the implant such that a tip of the actuator crosses the fracture and passes between the first bone fixator and the second bone fixator;
rotating the actuator within the implant to deploy one or more internal fixators within the first portion of the implant thereby securing the first portion of the implant to the proximal fragment; and
withdrawing the actuator from the implant after deploying the one or more internal fixators and prior to advancing the third bone fixator into the through hole of the implant,
wherein the through hole is within the second portion of the implant, and the third bone fixator is a bone screw that secures the second portion of the implant to the distal fragment.

10. The method of claim 7, further comprising:
advancing the first bone fixator across a first distance between a distal end of the first lumen and the distal fragment of the bone; and
advancing the second bone fixator across a second distance between a distal end of the second lumen and the distal fragment of the bone,
wherein the first distance and the second distance each range between about 0 mm and 20 mm.

11. The method of claim 7, further comprising:
moving the distal fragment away from the proximal fragment by applying the force.

12. The method of claim 7, further comprising:
rotating the distal fragment relative to the proximal fragment by applying the force.

13. The method of claim 1 wherein:
the first linear axis is offset from the second linear axis in both a lateral direction that is perpendicular to the first linear axis and a transverse direction that is perpendicular to both the first linear axis and the lateral direction; and
the first linear axis is offset from the third linear axis in both the lateral direction and the transverse direction.

14. A multi-lumen cannula comprising:
a proximal end, a distal end, and a body extending therebetween such that the body is symmetrical about a first plane;
a first lumen extending through the body along a first lumen axis parallel to and offset from the first plane;
a second lumen extending through the body along a second lumen axis parallel to the first lumen axis and offset from the first plane; and
a third lumen extending through the body along a third lumen axis parallel to the first lumen axis and positioned within the first plane,
wherein the first lumen axis and the second lumen axis are positioned within a second plane perpendicular to the first plane.

15. The multi-lumen cannula of claim 14 wherein the first lumen, the second lumen, and the third lumen each extend through the body from the proximal end to the distal end.

16. The multi-lumen cannula of claim 14 wherein body comprises two equal portions separated by the second plane.

17. The multi-lumen cannula of claim 14 wherein the second plane intersects the third lumen.

18. The multi-lumen cannula of claim 14 wherein the first lumen and the second lumen are positioned within the body between a third plane that perpendicularly intersects the first plane to form the third lumen axis and a fourth plane that is parallel to the third plane and tangent to the third lumen.

19. The multi-lumen cannula of claim 14 wherein the first and second lumens have cross-sectional shapes and the cross-sectional shape of the first lumen is the same as a cross-sectional shape of the second lumen.

20. The multi-lumen cannula of claim 19 wherein a maximum cross-sectional dimension of the third lumen is greater than a maximum cross-sectional dimension of the first lumen.

21. The multi-lumen cannula of claim 14 wherein the body has a non-circular cross-sectional shape along at least a portion of a length of the body measured parallel to the first lumen axis.

22. The multi-lumen cannula of claim 14 wherein the body defines a recess extending into the body from the proximal end toward the distal end, the recess defined such that the third lumen axis intersects the recess, wherein the cross-sectional shape of the recess is non-circular.

23. The multi-lumen cannula of claim 22 wherein the first lumen axis and the second lumen axis both intersect the recess.

24. The multi-lumen cannula of claim 14 wherein the first lumen defines a length measured along the first lumen axis and a width measured perpendicular to the first lumen axis, and the length is between 10 and 100 times greater than the width.

25. The multi-lumen cannula of claim 24 wherein when the length ranges between about 100 mm and 200 mm, and the width ranges between about 1 mm and 5 mm.

26. A multi-lumen cannula comprising:
a proximal end, a distal end, and a body extending therebetween;
a first lumen extending through the body from the proximal end to the distal end along a first lumen axis positioned within a first plane;
a second lumen extending through the body from the proximal end to the distal end along a second lumen axis that is parallel to the first lumen axis, positioned within the first plane, and offset from the first lumen axis in a first direction that is perpendicular to the first lumen axis; and
a third lumen extending through the body from the proximal end to the distal end along a third lumen axis that is parallel to the first lumen axis, positioned within a second plane that is parallel to the first plane, and offset from the first plane in a second direction that is perpendicular to the first plane,
wherein the first lumen and the second lumen are positioned between the second plane and a third plane that is parallel to the second plane and tangent to the third lumen.

27. The multi-lumen cannula of claim 26 wherein a cross-sectional shape of the first lumen perpendicular to the first lumen axis is the same as a cross-sectional shape of the second lumen perpendicular to the second lumen axis.

28. The multi-lumen cannula of claim 27 wherein a maximum cross-sectional dimension of the third lumen is greater than a maximum cross-sectional dimension of the first lumen.

29. The multi-lumen cannula of claim 26 wherein the first plane intersects the third lumen.

30. The multi-lumen cannula of claim 26 wherein the body has a non-circular cross-sectional shape along at least a portion of a length of the body measured parallel to the first lumen axis.

31. The multi-lumen cannula of claim 26, further comprising:
a recess extending into the body from the proximal end toward the distal end, the recess positioned such that the third lumen axis intersects the recess, wherein the cross-sectional shape of the recess is non-circular.

32. The multi-lumen cannula of claim 31 wherein the first lumen axis and the second lumen axis both intersect the recess.

33. The multi-lumen cannula of claim 26 wherein the first lumen defines a length measured along the first lumen axis and a width measured perpendicular to the first lumen axis, and the length is between 10 and 100 times greater than the width.

34. The multi-lumen cannula of claim 33 wherein when the length ranges between about 100 mm and 200 mm, and the width ranges between about 1 mm and 5 mm.

35. A multi-lumen cannula comprising:
a proximal end, a distal end, and a body extending therebetween;
a first lumen extending through the body along a first lumen axis, the first lumen having a first cross-sectional shape defined within a first plane perpendicular to the first lumen axis;
a second lumen extending through the body along a second lumen axis parallel to the first lumen axis, the second lumen having a second cross-sectional shape that is the same as the first cross-sectional shape and defined within the first plane; and
a third lumen extending through the body along a third lumen axis parallel to the first lumen axis, the third lumen having a third cross-sectional shape that is different from the first cross-sectional shape and defined within the first plane,
wherein the first lumen axis is offset from the second lumen axis in both a lateral direction that is perpendicular to the first lumen axis and a transverse direction that is perpendicular to both the first lumen axis and the lateral direction,
wherein the first lumen axis is offset from the third lumen axis in both the lateral direction and the transverse direction, and
wherein the third lumen axis is positioned within a second plane that is parallel to the lateral direction, and the first lumen axis and the second lumen axis are equidistant from the second plane with respect to the transverse direction.
